(19) United States
(12) Patent Application Publication
(10) Pub. No.: US 2026/0226382 A1
(43) Pub. Date: Aug. 6, 2026

(54) Device and Method for High Throughput 3D Cell Culture in a Well

(71) Applicant: BMF Precision, Inc., Maynard, MA (US)

(72) Inventor: Chunguang Xia, San Diego, CA (US)

(73) Assignee: BMF Precision, Inc., Maynard, MA (US)

(21) Appl. No.: 19/151,557

(22) PCT Filed: Feb. 2, 2024
(86) PCT No.: PCT/US2024/014143

§ 371 Date: Jul. 28, 2025

Related U.S. Application Data
(60) Provisional application No. 63/442,977, filed on Feb. 2, 2023.

(51) Int. Cl. C12M 1/32; C12M 1/00; C12M 3/06

(52) U.S. Cl. CPC C12M 23/12; C12M 23/16; C12M 23/38; C12M 29/10

(57) ABSTRACT

A 3D well-insert perfusion bioreactor system and method for more accurate modeling of in vivo environmental conditions and cellular behaviors. In some embodiments, the insert bioreactor includes an inlet coupled to a lower chamber and an outlet coupled to an upper chamber, where the chambers are separated by a perforated plate. A plurality of hairs extend downward from a lower surface of the insert bioreactor, where each hair includes a supply channel coupled to the lower chamber and a return channel coupled to the upper chamber. The supply channel of a hair is coupled to a return channel of the hair at a distal end of the hair. The distal end of the hair may include a membrane. In some embodiments, the insert bioreactor may be adapted to couple to a microfluidic system integrated within a culture plate.

### FIELD

This disclosure relates to cell biology and/or tissue engineering, and more particularly, to systems and methods for mimicking a 3D biological environment for cells via a 3D well-insert perfusion bioreactor as a 3D cell-culture model.

### BACKGROUND

Recent drug discovery efforts have focused on incorporating in vitro cell models that better mimic the in vivo conditions found within a target patient. Conventionally, a predetermined quantity or concentration of model cells is seeded onto a coated microplate well. The microplate is incubated to encourage the cells to attach in a two-dimensional (2D) monolayer before performing a prescribed assay. While this may provide some improvements over biochemical and immortalized cell lines, culturing cells in this 2D manner may be problematic, at least because 2D cell-culture models typically do not accurately model in vivo environmental conditions and/or cellular behaviors.

For example, studies of 2D cell-culture models have shown that attrition rates of drug candidates for cancer can be as high as approximately 95%. Further, in vitro drug efficacy values can fail to translate to clinical environments. Still further, unforeseen toxicity issues can arise. First, it is believed that in 2D cell-culture models, some extracellular matrix (ECM) components are missing, and therefore, certain cell-to-cell and cell-to-matrix interactions do not occur. These components and interactions are critical to cell differentiation, cell proliferation, and/or cellular function that normally occur in vivo. Second, it has been found that the environment of a 2D cell-culture model may not accurately mimic a three-dimensional (3D) in vivo environment where cancer cells reside, because the 2D environment does not allow for areas of hypoxia, heterogeneous cell populations, varying cell proliferation zones, ECM influences, soluble signal gradients, and/or differential nutrient and metabolic waste transport.

3D cell-culture models may be utilized to overcome many limitations of 2D cell-culture models because 3D cell-culture models more closely mimic the features and environments of complex in vivo conditions. For example, studies have shown that tumor cells of specific cell lines, evaluated using 3D cell-culture models, are less sensitive to anti-cancer agents than when the same tumor cells are cultured using 2D cell-culture models.

As the cell model moves from 2D to 3D, not only do cell interactions and expressions change dramatically, but also the kinetics of cellular metabolism varies. A fundamental challenge is to deliver the metabolites to the cells deep inside a bigger model (e.g., greater than 1 mm on an edge) and to transport waste and proteins out of the model. Hence, perfusion at microscale is desired.

One way to build 3D cell-culture models is to form 3D scaffolds out of porous polymeric and/or biologic materials. At least one study determined an optimal pore size (e.g., pore diameter) for 3D scaffolds of cell-culture models of approximately 100-400 μm. See Han, et. al, “Effect of Pore Size on Cell Behavior Using Melt Electrowritten Scaffolds,” Frontiers in Bioengineering and Biotechnology, Volume 9, Article 629270, July 2021. This result was based on achieving good cell nutrition, cell-growth space, and cell-scaffold interaction (the result depended on the specific cell lines).

Consistently controlling the size and shape of pores to achieve repeatable results requires a high precision manufacturing method. For example, to consistently create pores with diameters of 100 μm typically requires a manufacturing method having sub-20 μm resolution, such as micro 3D printing. In particular, projection micro stereolithography (PμSL) and two-photon polymerization are micro 3D printing techniques are capable of consistently creating complex 3D structures. Further, these printing techniques can create 3D structures from biocompatible and/or biodegradable polymers, such as poly-ethylene glycol (PEG) and poly lactic acid (PLA). After polymerization, these polymers may be hard (e.g., greater than 1 GPa elastic modulus) or soft (e.g., less than 1 MPa elastic modulus).

Cell cultures can be described in terms of reaction kinetics. In steady state, the process of cells consuming metabolites (e.g., cell nutrients) is often described by the Michaelis-Menten equation:

$$v = \frac{{V}_{max} [ S ]}{{K}_{M} + [ S ]} ,$$

where v is the velocity of the reaction, Vₘₐₓ is the maximum uptake rate of metabolites, K_{M} is the metabolite concentration when the uptake rate is half of the maximum (the Michaelis constant), and [S] is the concentration of metabolites. In Michaelis-Menten kinetics, the consumption behavior follows first order kinetics at low concentration. This means the consumption rate is proportional to the concentration. As the concentration of the metabolite increases, the consumption behavior will gradually become zero order kinetics. This means the consumption rate is near or equal to the maximum velocity and is independent of metabolite concentration. This is because the cells eventually become saturated and therefore their intake of metabolites reaches a plateau.

Metabolites are depleted as they transport through a cell cluster/tissue. The consumption rate decreases approximately parabolically. In an in vivo environment, when the cell concentration is on the order of 10¹⁰ cells/ml, the cells stay less than 100 μm from blood capillaries. It is therefore advantageous for cells to stay within the zone that the nutrient concentration is higher than K_{M}, (the metabolite concentration when the uptake rate is half of the maximum). It has been shown that at a high cell density, for 10 μm capillary tube wall thickness with metabolite diffusivity of 10-9 cm²/s, the nutrient concentration decreases over 80% after going through the wall of the tube. See Xia, “3D Microfabricated Bioreactor with Capillaries,” Biomedical Microdevices 11 (6): 1309-1315 October 2009. Hence, the perfusion culture media will need higher concentration than in traditional 2D direct media contact culture. To save on the cost of media, especially in high throughput mass drug screening processes, a higher density of nutrient transport passages (e.g., capillaries), that can support medias of lower concentration, can be utilized to emulate a higher media concentration.

At the early stage of a new drug development, tens of thousands of compounds are screened. This process typically requires a high throughput method for compound screening. In current 2D cell-culture methods, typically 96-well or 384-well culture plates are utilized. Hence any new 3D cell-culture model should not only be more accurate than current 2D methods, but it should be able to accommodate high throughput processes for screening large amounts of potential chemical compounds for targeted treatment effects. However, current 3D perfusion models lack such high throughput capability due in part to physical size limitations and issues with system integration.

In a human body, organ systems communicate with each other in certain ways to keep the body in balance, where some communications are in series and others are in parallel. Communication among organ systems is controlled mainly by the autonomic nervous system and the endocrine system. The nervous system transmits signals by electrical pulses, and the endocrine system carries signal with hormones in blood vessels and via other fluidic channels.

Some perfusion models seek to replicate or emulate endocrine-like organ-system communications via microfluidic channels. However, due in part to limitations in current manufacturing capabilities, a high density (96 organoid per culture plate or higher), three dimensional (e.g., greater than 1 mm thick), and high throughput perfusion organoid system does not exit. Embodiments of the disclosed invention overcome several of the above-described limitations.

### SUMMARY

The disclosed embodiments provide for a perfusion well insert (scaffold) for a 3D cell culture with a capillary system to promote 3D cell-to-cell and cell-to-ECM interactions, and at the same time to promote the transportation of metabolites and metabolic waste for high-cell-density 3D cell cultures. In some embodiments, the well inserts conform to the dimensions of the wells in standard multi-well culture plates. In some embodiments, an array assembly of such well inserts may be connected by microfluidic networks to provide a high throughput system that is compatible with any multi-well culture plate. Such a perfusion well insert (scaffold) can better mimic the real biological environment of cells in vivo, which is critical for an accurate drug screening, without sacrificing the high throughput of traditional 2D culture plate screening.

In one embodiment, the 3D well-insert perfusion bioreactor includes:
an inlet fluidically coupled to a first chamber and an outlet fluidically coupled to a second chamber; and
a plurality of hairs extending downward from a lower surface of the bioreactor, each hair comprising a supply channel fluidically coupling the first chamber to a tip of the hair and a return channel fluidically coupling the second chamber to the tip of the hair, wherein the supply channel and the return channel are fluidically coupled at the tip of the hair.

In some embodiments there is a perforated plate disposed between the first chamber and the second chamber, where the first chamber is gravitationally below the second chamber.

In some embodiments, the lower surface of the bioreactor forms a downward-pointing cone.

In some embodiments, inlet and the outlet are disposed within an elongated member adapted to physically engage with a culture-plate cover and to fluidically couple to a microfluidic system integrated within the culture-plate cover, where the microfluidic system includes:
a cover inlet fluidically coupled to a cover inlet distribution channel that is adapted to fluidically couple to the inlet; and
a cover outlet fluidically coupled to a cover outlet distribution channel that is adapted to fluidically couple to the outlet.

In some embodiments, an input culture media is pumped into the inlet, and the outlet is fluidically coupled to a recycling system.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings.

FIG. 1A shows a perspective view of an exemplary 3D well insert from a side and FIG. 1B shows a perspective view of the exemplary 3D well insert from below.

FIGS. 2A-2C show various views of one or more hairs of an exemplary 3D well insert.

FIGS. 3A-3C show various cutaway views of an exemplary 3D well insert.

FIG. 4A shows a view of a lower chamber of an exemplary 3D well insert and FIGS. 4B-4C show various cutaway views thereof.

FIG. 5A shows an exemplary 96-well culture plate, FIG. 5B shows 48 exemplary 3D well inserts adjacent to corresponding wells of the culture plate, and FIG. 5C shows the 48 exemplary 3D well inserts inserted into corresponding wells of the culture plate.

FIG. 6A shows a bottom perspective view of 48 exemplary 3D well inserts installed into corresponding holes of an exemplary 96-well cover plate, FIG. 6B shows a side perspective view thereof, and FIG. 6C shows an exemplary distribution channel of the cover plate.

FIG. 7 shows a side perspective view of an exemplary cover plate with 48 exemplary 3D well inserts engaged thereto being lowered onto an exemplary 96-well culture plate.

FIG. 8A diagrams Boolean AND gate functionality achieved by an embodiment and FIG. 8B diagrams Boolean OR gate functionality achieved by an embodiment.

### DETAILED DESCRIPTION

The present disclosure may be more readily understood by reference to the following detailed description and the accompanying drawings, which form a part of this disclosure. This disclosure is not limited to the specific devices, methods, conditions, or parameters described and/or shown herein, and the terminology used herein is for the purpose of describing particular embodiments by way of example only and is not intended to be limiting of disclosed embodiments or inventions. For example, “left,” “right,” “clockwise,” and “counterclockwise” may be used as specific examples of generally opposite lateral or rotational directions, respectively. Also, as used in the specification and including the appended claims, the singular forms “a,” “an,” and “the” include the plural, and reference to a particular numerical value includes at least that particular value, unless the context clearly dictates otherwise.

The following numerals are used to describe various features of the embodiments. 3D well insert (abbreviated as “insert”) 100; inlet 110; outlet 120; distribution chamber 200; upper chamber 210; lower chamber 220; wetting cone 230; diverter outlet 240; inlet tube 250; diverter tube 260; isolation tube 270; hair injection hole 280; perforated plate 290; hair 300; supply channel 310; return channel 320; interconnect 330; membrane 340; support 350; microfluidic system 400; cover 410; cover inlet 420; cover outlet 430; cover inlet distribution channel 440; cover outlet distribution channel 450; cover channel separation wall 460.

There are a few types of cell-culture plates available in the biomedical industry that vary based on a number and configuration of wells therein, for example 6-well, 12-well, 24-well, 48-well, 96-well, 384-well, and so on. For high throughput screening, the industry currently tends to use 96-well or 384-well plates or larger. Culture plates can be further divided by the shape of the well, such as flat-bottom, round-bottom, V-bottom, and so on. While the disclosed invention covers any size and shape of culture plate, the embodiments described in detail focus on an exemplary 96-well flat-bottom plate for simplicity.

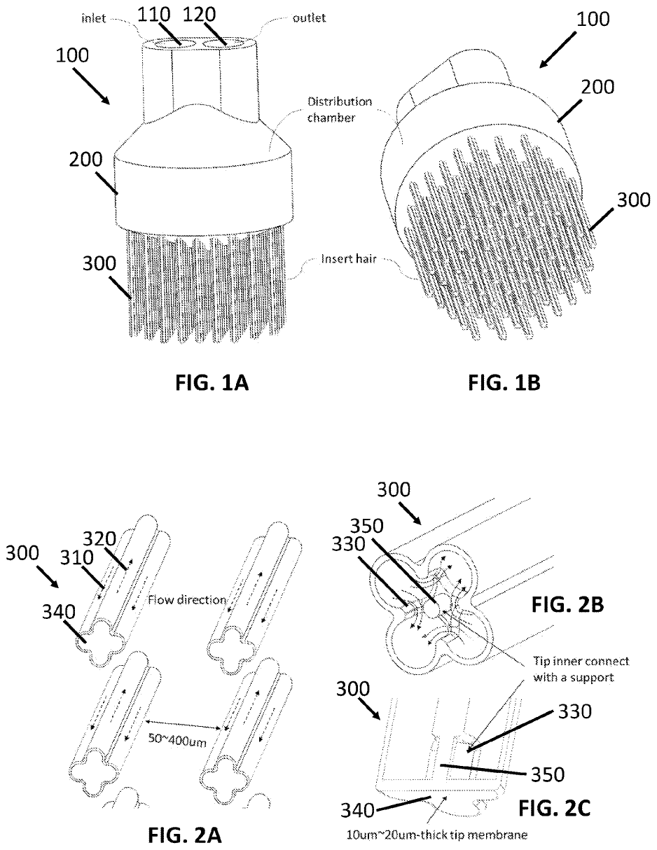

FIGS. 1A-1B shows a 3D well insert 100 having a distribution chamber 200, an inlet 110, and outlet 120, and a plurality of hairs 300. The inlet 110 and outlet 120 are parallel and are both connected to the distribution chamber 200. In this embodiment, the inlet 110 and the outlet 120 are adjacent and parallel to each other to achieve an efficient integration with a culture media delivery microfluidic network that is described in later herein. Other relative arrangements of the inlet 110 and outlet 120 are possible, including a coaxial configuration where the inlet 110 is surrounded by a ring-shaped outlet 120 (or vice versa). Culture media that enters the distribution chamber 200 (e.g., via the inlet 110) is distributed to the hairs 300 via the distribution chamber 200. Culture media that returns from the hairs 300 enters the distribution chamber 200 and may flow out of the insert 100 via the outlet 120.

In one embodiment, the outer diameter of the insert 100 is 6.4 mm, which is chosen to be compatible with (fit inside of) the wells of a standard 96-well culture plate. In one embodiment, the length of the hairs 300 may vary from 3 to 4 mm across the cluster of hairs 300, such that hairs 300 at or near the center of the cluster are the shortest and the hairs 300 gradually become longer as they get closer to the outer edge of the cluster.

The insert 100 shown in FIG. 1A-1B is configured for a flat bottom well, such that the hairs 300 end at a same plane that is parallel to the bottom of a well. In some embodiments, the plane is approximately 50-200 μm above the bottom of the well (there is a 50-200 μm gap between the distal ends (tips) of the hairs 300 and the bottom of the well). The length of the hairs 300 may be adjusted according to the profile of a well bottom, for example, for round-bottom wells or V-bottom wells, such that there is the same gap dimension between the distal end of each hair 300 and the bottom of the well.

In one embodiment, the total number of hairs in an insert 100 is 69, which may be arranged in a linear array with a pitch (spacing between adjacent hairs 300) of 400 μm, and the hairs 300 may be placed at a density of 2 hairs/mm². The pitch may be reduced to accommodate different cell lines and may typically be in the range of 50-400 μm as indicated in FIG. 2A. Other patterns, arrangements, or configurations of the hairs 300 may be utilized, including, for example, non-uniform pitch, randomized pitch in randomized directions, circular pattern, Fibonacci pattern, and so on. The number of hairs 300 of an insert 100 may be proportion to the size of a corresponding well, and the density of hairs 300 may be constant or they may vary with the size of an insert 100. For example, the total number of hairs 300 of an insert 100, configured for use in a standard 384-well plate, may be around 15 hairs at a density of 2 hairs/mm².

FIGS. 2A-2B show close-up views of segments of one or more hairs 300 and FIG. 2C shows a longitudinal cross section of a distal end (e.g., tip) of a hair 300. In the shown embodiment, each hair 300 comprises four longitudinal channels that resemble an “x” or “+” sign when viewed in transverse cross section (FIGS. 2A-2B). Two of the channels are supply channels 310 that deliver culture media towards or into a well, and two of the channels are return channels 320 that remove culture media away from or out of the well. In one embodiment, each channel has a circular cross-section, has an inner diameter of approximately 80 μm, and has an outer diameter of approximately 100 μm. In one embodiment, the area between the four centers of the four channels (two supply channels 310 and two return channels 320) forms a square with edges of approximately 100 μm each.

One advantage of the “x” or “+” shaped cross section of a hair as shown in FIGS. 2A-2B is to achieve a certain strength of the hair 300 without sacrificing the surface area of the hair 300, which is important for the exchange of certain substances through the outer walls of the hair 300, especially: (1) the diffusion of nutrients from fresh culture media flowing in the supply channels 310 to the well where they may be consumed or otherwise utilized by cells in the well outside of the hair 300; and (2) the diffusion of metabolic waste from the cells in the well to the return channels 320 where they may be carried away. If the concentration of metabolic waste is too high (e.g., 100 μmol/L of nitrogen compounds), it may be toxic to the health of the cells in the well. Therefore, it is important to remove the wastes as the metabolism continues. In some embodiments, the walls of some or all of the hairs 300 may be perforated with micro holes to increase the diffusion of certain substances through the walls of the hairs 300 for both nutrients and wastes (e.g., proteins and other molecules). In some embodiments, the micro holes may have a diameter of approximately 0.05-5 μm.

As shown in FIGS. 2B-2C, all the four channels merge at the tip of a hair 300 at an interconnect 330. At or adjacent to a distalmost end of the tip of a hair 300 there is a membrane 340 that isolates fluid in the channels of the hair 300 from fluid in the well. In some embodiments, the membrane 340 may be approximately 5-20 μm thick. To increase the rigidity of the membrane 340, a support 350 may be utilized to hold the membrane 340 at or near a centroid of the membrane 340. In some embodiments, the support 350 is a cylindrical bar extending from a central axis of the hair 300 having a diameter of approximately 20 μm.

Some embodiments of the insert 100 utilize different configurations for the channels of each hair 300, for example a hair 300 may have only two channels consisting of one supply channel 310 and one return channel 320. As another example, a hair 300 may have four channels consisting of three supply channels 310 and one return channel 320.

In some embodiments, the insert 100 should comprise class II biocompatible materials or higher, for example PEG (polyethylene glycol). Some materials may require surface treatment for cell adhesion, such as a poly-L-lysine solution, to coat the surface of the material to achieve adequate cell attachment thereto, in the case of cell-culture experiments that require model cells to attach and proliferate on the hairs 300.

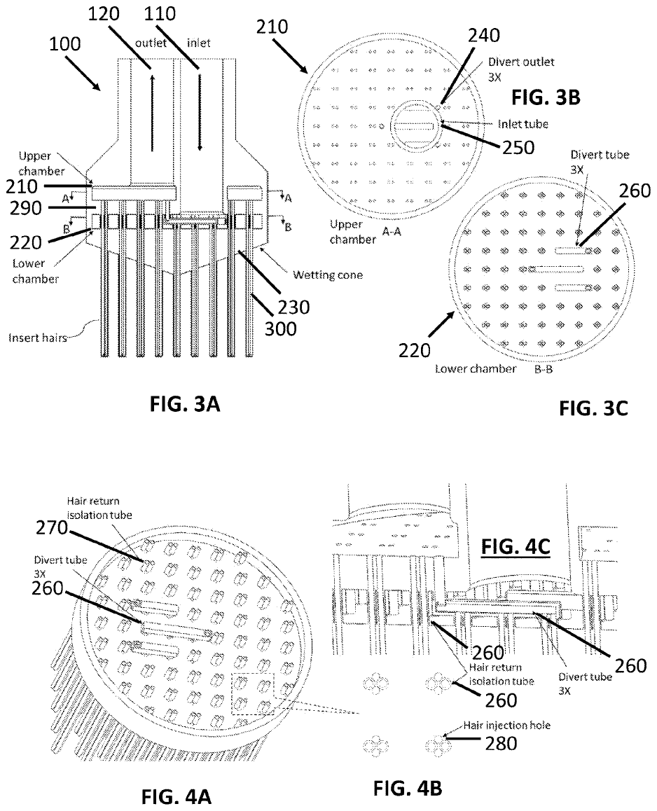

FIGS. 3A-3C show the internal structure of an embodiment of the insert 100. The distribution chamber 200 is divided into a lower chamber 220 and an upper chamber 210, with the upper chamber 210 gravitationally above the lower chamber 220 and separated by a perforated plate 290. In some embodiments, the upper chamber 210 and the lower chamber 220 are each approximately 500 μm tall (e.g., a height in a vertical direction), and the perforated plate 290 is approximately 500 μm thick. As shown in FIG. 3A, the inlet 110 of the insert 100 traverses through the upper chamber 210 and is directly connected or coupled to a roof of the lower chamber 220.

Culture media flowing into the inlet 110 will fill the lower chamber 220 and will be distributed into the supply channels 310, which are connected or coupled to a bottom of the lower chamber 220, via the hair injection holes 280 as shown in FIG. 4B. Once injected into the hairs 50, the culture media may flow gravitationally downward in the supply channels 310 toward the tip of each hair 300 (“supply flow”). At the tip of each hair 300, culture media may flow from the supply channels 310 into the return channels 320 via the interconnect 330. Once in the return channels 320, the culture media may flow gravitationally upward (“return flow”). In the embodiment shown in FIG. 3A, where the upper chamber 210 is immediately above the lower chamber 220, the return flow must go through the lower chamber 220 to get to the upper chamber 210.

To isolate the supply flow and the return flow from each other, and to form a complete flow loop to and from the tip of a hair 300, isolation tubes 270 may be disposed at least partly within the lower chamber 220 as shown in FIG. 4A. Each isolation tube 270 traverse through the lower chamber 220, having one end connected to a return channel 320 and another end connected to the upper chamber 210 via the perforated plate 290. The return flows from each hair 300 meet in the upper chamber 210 and converge in the outlet 120.

As shown in FIG. 4A-4C, the inlet 110 traverses the upper chamber 210 and connects or couples to a roof of the lower chamber 220. In some embodiments, the inlet 110 (surrounded by a tube) cuts through the upper chamber 210 to reach the lower chamber 220. Thus, the return channels 320 encompassed by the inlet 110 are unable to directly reach the upper chamber 210. In this case, these return channels 320 are connected to horizontal divert tubes 260 as shown in FIGS. 3A-3C, FIG. 4A, and FIG. 4C. These diverter tubes 260 collect the return flows from the seven hairs 300 encompassed within the inlet 110, and horizontally shift the return flows thereof away from the interior of the inlet, for example, to just beyond the outer edge of the inlet 110. The diverted flow may be released to the upper chamber 210 through three diverter outlets 240 each at or near an end of a diverter tube 260. In some embodiments, the diverter tubes 260 are disposed within the perforated plate 290. In some embodiments, each diverter tube 260 has an outlet diameter of approximately 150 μm. In some embodiments, the cross-sectional area of the inlet 110 is large enough to encompass seven hairs 300 (as shown in FIGS. 3A-3C, 4A, and 4C). The return channels 320 of these seven hairs are not connected to the upper chamber 210 as are the return channels 320 of the other hairs 300. Instead, as indicated above, these seven return channels 320 are connected to three horizontal divert tubes 260 as shown in FIGS. 3A-3C, FIG. 4A, and FIG. 4C.

When inserts 100 are inserted into corresponding wells of a culture plate, the wells may contain cells mixed with culture media, hydrogels, or extra cellular matrix (ECM). Therefore, to reduce the formation of bubbles or the entrapment of air or other gases when inserting the inserts 100, each insert 100 may be configured with a downward-pointing wetting cone 230 that is disposed gravitationally above the hairs 300, i.e., adjacent to the lower chamber 220, as shown in FIG. 3A. The wetting cone 230 assists in pushing bubbles of air and other gases within the well (having fluid therein) towards an outer edge of an insert 100 when the insert 100 is inserted into the well, thereby allowing the bubbles to escape the well more easily. In some embodiments, the wetting cone 230 is a right cone having a vertex angle of approximately 60-75 degrees (where the vertex angle is the angle between a cone's axis and the slanted surface). In other words, the wetting cone 230 may form a downward-facing pointed tip having an angle of approximately 120-150 degrees between opposite faces of its slanted surface.

As indicated above, certain dimensions of each insert 100 may conform to the size of a well in a culture plate. In some embodiments, the outer diameter of an insert 100 may be slightly smaller than the inside diameter of a well, such that when the insert 100 is inserted in the well, there may be a gap between a side wall of the well and a side wall of the insert 100 which may promote the exchange or release of gas from the well. In some embodiments, the gap may be approximately 50-150 μm.

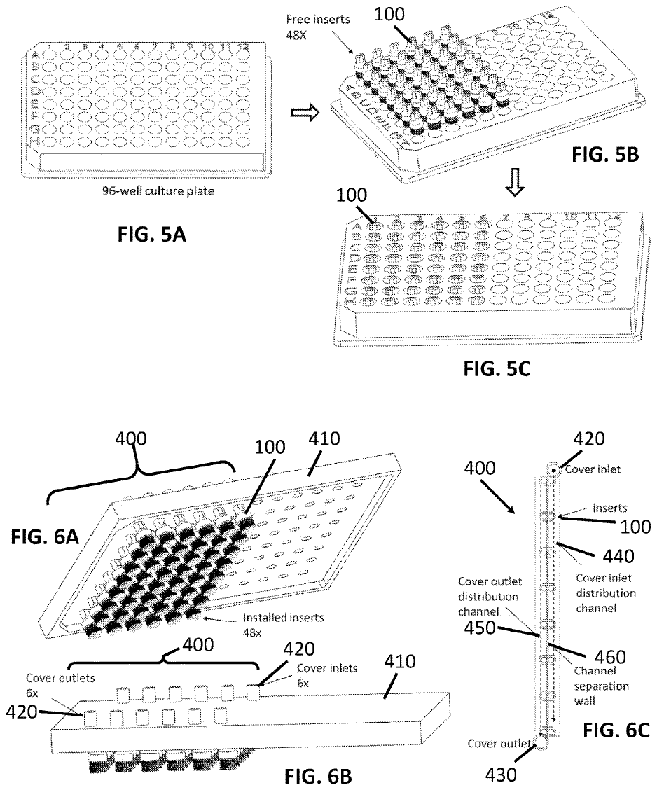

An array of inserts 100 may be placed in a multi-well culture plate when carrying out cell culture processes. The size of an array of inserts 100 may vary up to the maximum number of wells in a culture plate, and the array may be dense, sparse, uniform, or non-uniform. For example, an 8×12 array may comprise up to 96 inserts 100, or it may consist of less than 96. FIG. 5A shows an exemplary 96-well culture plate. FIG. 5B shows a dense and uniform 8×6 array of inserts 100 comprising 48 inserts 100 adjacent to the culture plate. FIG. 5C shows the array of inserts 100 inserted into corresponding wells of the culture plate. In some embodiments, when the inserts 100 are inserted, the tips of the hairs 300 may be approximately 50-200 μm from the bottom of wells. Such a gap may promote cells that are located at or near the bottom of the well to attach to and proliferate on the hairs 300. Wells of a culture plate without inserts 100 inserted therein, for example the 48 empty wells shown in FIGS. 5B-SC, may be used as control wells, e.g., to compare the results of cell cultures with and without the aid of an insert 100.

In some embodiments, the inserts 100 may provide a 3D cell-culture model that mimics the in vivo conditions for studying cell biology and tissue engineering, for example high-density cell-to-cell interaction, high-density cell-to-ECM interaction, high-density cell-to-drug response, bio-material development, and new drug development. When performing cell-culture processes, the wells of a culture plate are typically prefilled with certain amount of a cell-media-hydrogel mixture fluid, and this amount may be controlled so that when the inserts 100 are inserted in the wells, the mixture fluid will not overflow above the upper chamber 210. The mixture fluid composition and/or amount of prefilling thereof may be different for each well.

In some embodiments, the inlet 110 and the outlet 120 of each insert 100 may be coupled to an external pumping and recycling system, respectively, to activate a perfusion functionality within each insert 100. A high density of the small inserts 100 (e.g., greater than 1/cm²) may require a microfluidic system 400 that can gently deliver the culture media to the inserts 100. In some embodiments, the microfluidic system 400 can group any combination of the wells in the culture plate together to receive a given type or version of culture media. In some embodiments, the microfluidic system 400 may be integrated with a cover 410 of a culture plate as shown in FIG. 6A-6B. The inserts 100 may be inserted into or coupled to holes within the cover 410 first, then the cover 410 may be placed on the culture plate. In some embodiments, each column of eight wells of a culture plate may be considered as one group. One cover inlet 420 that is disposed adjacent to a first edge of the cover 410 may supply (deliver) media to this group, and one cover outlet 430 that is disposed on an opposite edge of the cover 410 may collect (return) media from this group. In other embodiments, the relative positions of the cover inlet 420 and cover outlet 430 may vary.

The cover inlet 420 and the cover outlet 430 may be fluidically coupled to two channels, a cover inlet distribution channel 440 and a cover outlet distribution channel, respectively as shown in FIG. 6C. In this embodiment, the cover inlet distribution channel 440 is couped to all eight inlets 110 of the inserts 100 within this group, and the cover outlet distribution channel 450 is coupled to all eight outlets 120 of the inserts 100 of this group. The cover inlet distribution channel 440 and the cover outlet distribution channel 450 are adjacent to each other and are separated by a cover channel separation wall 460 as shown in FIG. 6C. In some embodiments, the cover inlet distribution channel 440 and the cover outlet distribution channel 450 each have approximately a 3×2 mm cross section and the cover channel separation wall has a thickness of approximately 250 μm.

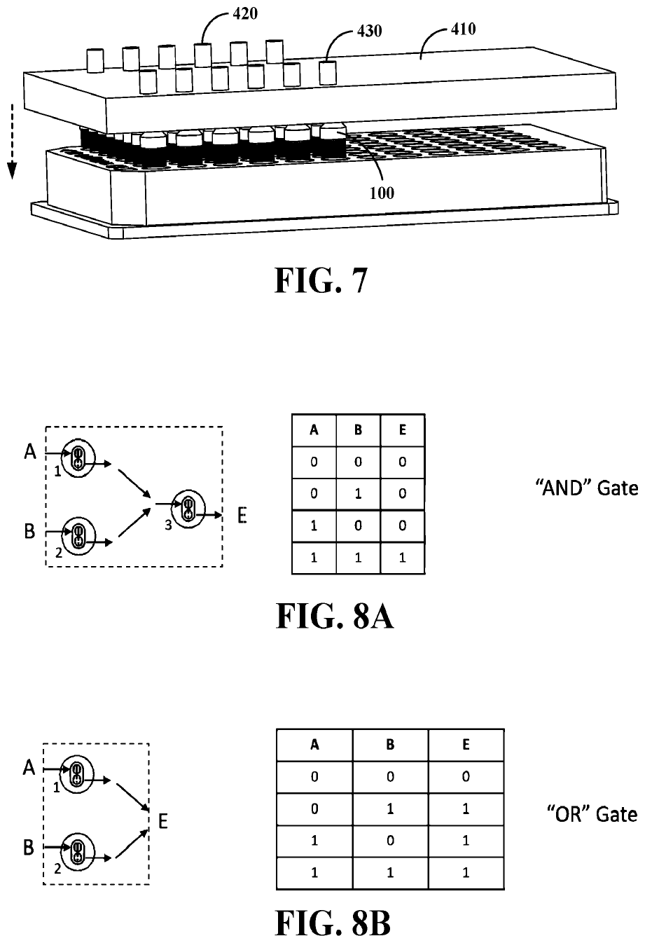

In some embodiments, the cover inlets 420 may be fluidically coupled to an external pumping system, and the cover outlets 430 may be fluidically coupled to a recycling system. When the cover 410 has been prepared, for example by inserting inserts 100 therein, the cover 410 may be slowly lowered onto a culture plate with the inserts 100 aligned with the wells as shown in FIG. 7. The assembled cover 410 and culture plate may then be placed in a cell-culture incubator, for example, where humidity and CO₂ levels are controlled.

One advantage of integrating the microfluidic system 400 within the cover 410 for a culture plate comprising a grid of wells is that interactions between certain wells can be programmed via Boolean functionality, for example logical AND and logical OR. If each well represents an organoid with specific organ functions, then it is possible to build a more complicated “body” model comprising multiple organoids by using these basic logical gates as building blocks. FIG. 8A depicts a logical AND gate. Chemical “A” is delivered into well “1” and chemical “B” is delivered into well “2,” each via the microfluidic system 400. Chemical “E” is released from well “3” via the microfluidic system 400 only when products from well “1” (after receiving chemical “A”) and from well “2” (after receiving chemical “B”) reach and react with cells in well “3.” The corresponding logical AND functionality is depicted in the truth table of FIG. 8A. FIG. 8B depicts a logical OR gate. When either chemical “A” is delivered to well “1” or chemical “B” is delivered to well “2,” each via the microfluidic system 400, chemical “E” is released. The corresponding logical OR functionality is depicted in the truth table of FIG. 8B.

Some implementations of high throughput 3D cell culture in a well disclosed herein include a 3D well-insert perfusion bioreactor, comprising: an inlet fluidically coupled to a first chamber and an outlet fluidically coupled to a second chamber; and a plurality of hairs extending downward from a lower surface of the bioreactor, each hair comprising a supply channel fluidically coupling the first chamber to a tip of the hair and a return channel fluidically coupling the second chamber to the tip of the hair, wherein the supply channel and the return channel are fluidically coupled at the tip of the hair.

In some implementations, the bioreactor further comprises a perforated plate disposed between the first chamber and the second chamber, wherein the first chamber is gravitationally below the second chamber.

In some implementations, the bioreactor further comprises a plurality of isolation tubes each vertically traversing the first chamber and fluidically isolating a return channel from the first chamber.

In some implementations, the bioreactor further comprises one or more diverter tubes disposed at least partly within the perforated plate and extending from at least one return channel encompassed by the inlet to the second chamber.

In some implementations, the lower surface of the bioreactor forms a downward-pointing cone.

In some implementations, the bioreactor further comprises a membrane at a distal end of each hair.

In some implementations, the hair comprises a support therein that is attached to the membrane near a centroid thereof.

In some implementations, the inlet and the outlet are disposed within an elongated member adapted to physically engage with a culture-plate cover and to fluidically couple to a microfluidic system integrated within the culture-plate cover, wherein the microfluidic system comprises: a cover inlet fluidically coupled to a cover inlet distribution channel that is adapted to fluidically couple to the inlet; and a cover outlet fluidically coupled to a cover outlet distribution channel that is adapted to fluidically couple to the outlet.

Some implementations of high throughput 3D cell culture in a well disclosed herein include a method of culturing cells, comprising immersing a 3D well-insert perfusion bioreactor into a cell-culture-media mixture contained within a well of a culture plate, the bioreactor comprising: an inlet fluidically coupled to a first chamber and an outlet fluidically coupled to a second chamber; and a plurality of hairs extending downward from a lower surface of the bioreactor, each hair comprising a supply channel fluidically coupling the first chamber to a tip of the hair and a return channel fluidically coupling the second chamber to the tip of the hair, wherein the supply channel and the return channel are fluidically coupled at the tip of the hair; and incubating the cells.

In some implementations, the method further comprises pumping an input culture media into the inlet.

In some implementations, the method further comprises fluidically coupling the outlet to a recycling system.

In some implementations, the bioreactor further comprises a perforated plate disposed between the first chamber and the second chamber, wherein the first chamber is gravitationally below the second chamber.

In some implementations, the bioreactor further comprises a plurality of isolation tubes each vertically traversing the first chamber and fluidically isolating a return channel from the first chamber.

In some implementations, the bioreactor further comprises one or more diverter tubes disposed at least partly within the perforated plate and extending from at least one return channel encompassed by the inlet to the second chamber.

In some implementations, the lower surface of the bioreactor forms a downward-pointing cone.

In some implementations, the bioreactor further comprises a membrane at a distal end of each hair.

In some implementations, the hair comprises a support therein that is attached to the membrane near a centroid thereof.

In some implementations, the inlet and the outlet are disposed within an elongated member adapted to physically engage with a culture-plate cover and to fluidically couple to a microfluidic system integrated within the culture-plate cover, wherein the microfluidic system comprises: a cover inlet fluidically coupled to a cover inlet distribution channel that is adapted to fluidically couple to the inlet; and a cover outlet fluidically coupled to a cover outlet distribution channel that is adapted to fluidically couple to the outlet.

In some implementations, the method further comprises physically engaging the bioreactor with a culture-plate cover and fluidically coupling the inlet and the outlet to a microfluidic system integrated within the culture-plate cover, wherein the microfluidic system comprises: a cover inlet fluidically coupled to a cover inlet distribution channel that is adapted to fluidically couple to the inlet; and a cover outlet fluidically coupled to a cover outlet distribution channel that is adapted to fluidically couple to the outlet.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

1. A 3D well-insert perfusion bioreactor, comprising:
an inlet fluidically coupled to a first chamber and an outlet fluidically coupled to a second chamber; and
a plurality of hairs extending downward from a lower surface of the bioreactor, each hair comprising a supply channel fluidically coupling the first chamber to a tip of the hair and a return channel fluidically coupling the second chamber to the tip of the hair, wherein the supply channel and the return channel are fluidically coupled at the tip of the hair.

2. The bioreactor of claim 1 further comprising a perforated plate disposed between the first chamber and the second chamber, wherein the first chamber is gravitationally below the second chamber.

3. The bioreactor of claim 2 further comprising a plurality of isolation tubes each vertically traversing the first chamber and fluidically isolating a respective return channel from the first chamber.

4. The bioreactor of claim 3 further comprising one or more diverter tubes disposed at least partly within the perforated plate and extending from at least one return channel encompassed by the inlet to the second chamber.

5. The bioreactor of claim 1 wherein the lower surface of the bioreactor forms a downward-pointing cone.

6. The bioreactor of claim 1 further comprising a membrane at a distal end of each hair.

7. The bioreactor of claim 6 wherein the hair comprises a support therein that is attached to the membrane near a centroid thereof.

8. The bioreactor of claim 1 wherein the inlet and the outlet are disposed within an elongated member adapted to physically engage with a culture-plate cover and to fluidically couple to a microfluidic system integrated within the culture-plate cover, wherein the microfluidic system comprises:
a cover inlet fluidically coupled to a cover inlet distribution channel that is adapted to fluidically couple to the inlet; and
a cover outlet fluidically coupled to a cover outlet distribution channel that is adapted to fluidically couple to the outlet.

9. A method of culturing cells, comprising:
immersing a 3D well-insert perfusion bioreactor into a cell-culture-media mixture contained within a well of a culture plate, the bioreactor comprising:
an inlet fluidically coupled to a first chamber and an outlet fluidically coupled to a second chamber; and
a plurality of hairs extending downward from a lower surface of the bioreactor, each hair comprising a supply channel fluidically coupling the first chamber to a tip of the hair and a return channel fluidically coupling the second chamber to the tip of the hair, wherein the supply channel and the return channel are fluidically coupled at the tip of the hair; and incubating the cells.

10. The method of claim 9 further comprising pumping an input culture media into the inlet.

11. The method of claim 10 further comprising fluidically coupling the outlet to a recycling system.

12. The method of claim 9 wherein the bioreactor further comprises a perforated plate disposed between the first chamber and the second chamber, wherein the first chamber is gravitationally below the second chamber.

13. The method of claim 12 wherein the bioreactor further comprises a plurality of isolation tubes each vertically traversing the first chamber and fluidically isolating a respective return channel from the first chamber.

14. The method of claim 13 wherein the bioreactor further comprises one or more diverter tubes disposed at least partly within the perforated plate and extending from at least one return channel encompassed by the inlet to the second chamber.

15. The method of claim 9 wherein the lower surface of the bioreactor forms a downward-pointing cone.

16. The method of claim 9 wherein the bioreactor further comprises a membrane at a distal end of each hair.

17. The method of claim 16 wherein the hair comprises a support therein that is attached to the membrane near a centroid thereof.

18. The method of claim 9 wherein the inlet and the outlet are disposed within an elongated member adapted to physically engage with a culture-plate cover and to fluidically couple to a microfluidic system integrated within the culture-plate cover, wherein the microfluidic system comprises:
a cover inlet fluidically coupled to a cover inlet distribution channel that is adapted to fluidically couple to the inlet; and
a cover outlet fluidically coupled to a cover outlet distribution channel that is adapted to fluidically couple to the outlet.

19. The method of claim 9 further comprising physically engaging the bioreactor with a culture-plate cover and fluidically coupling the inlet and the outlet to a microfluidic system integrated within the culture-plate cover, wherein the microfluidic system comprises:
a cover inlet fluidically coupled to a cover inlet distribution channel that is adapted to fluidically couple to the inlet; and
a cover outlet fluidically coupled to a cover outlet distribution channel that is adapted to fluidically couple to the outlet.

20. The bioreactor of claim 1, wherein each hair comprises four channels that resemble an “x” or “+” sign when viewed in transverse cross section, wherein two of the four channels are supply channels and two of the four channels are return channels.
